(19) United States
(12) Patent Application Publication
(10) Pub. No.: US 2013/0115176 A1
(43) Pub. Date: May 9, 2013

(54) MATERIALS FOR DISSOLUTION-CONTROLLED RELEASE

(71) Applicant: Board of Trustees at Southern Illinois University, Carbondale, IL (US)

(72) Inventor: Bakul C. Dave, Carbondale, IL (US)

(73) Assignee: Board of Trustees at Southern Illinois University, Carbondale, IL (US)

(21) Appl. No.: 13/658,635

(22) Filed: Oct. 23, 2012

Related U.S. Application Data
(63) Continuation of application No. 12/180,561, filed on Jul. 28, 2008.
(60) Provisional application No. 60/952,164, filed on Jul. 26, 2007.

(51) Int. Cl. C11D 7/32; C09B 67/02; A61K 47/24

(52) U.S. Cl. CPC C11D 7/3209; A61K 47/24; C09B 67/0097
USPC 424/59; 514/788; 510/347; 510/515; 512/2; 8/581; 424/94.1; 514/18.8

(57) ABSTRACT

A controlled release substance containing organosilica sol-gels is provided. The organosilica sol-gels may be further functionalized with amino groups to control the rate of release of encapsulated molecules. The controlled release substance may be utilized as a controlled release fabric care substance, including at least one active fabric care substance in a host matrix comprising an organosilica gel. The fabric care substance may be at least one of a protein, enzyme, fragrance, anti-static agent, fabric softener, dye, or detergent, and may be in bead form, particle form or powder form.

### CROSS-REFERENCE TO RELATED APPLICATIONS

This application is a continuation of U.S. patent application No. 12/180,561, filed Jul. 28, 2008, which claims the benefit of U.S. Provisional Patent Application Serial No. 60/952,164, filed on Jul. 26, 2007, the entire disclosures of each are incorporated herein by reference.

### FIELD

The present disclosure relates to controlled release substances with controlled dissolution rates.

### BACKGROUND

The statements in this section merely provide background information related to the present disclosure and may not constitute prior art.

At present, enzymes are widely used in the detergent industry, and proeases added to detergents bring about hydrolysis of the peptide linkages in protein-based stains. Preventing the denatuation of the protein molecule in the multicomponent detergent environment presents considerable challenges. The problem is further heightened when mixtures of enzymes are used in detergents to serve different purposes. As a result, there is a need for matrixes that can be used for immobilization, stabilization and fast release of the proteases when placed in a water-rich environment.

### SUMMARY

In one aspect of the present disclosure, various embodiments of a controlled release substance containing organosilica sol-gels are provided. The organosilica sol-gels may be further functionalized with amino groups to control the rate of release of encapsulated molecules.

In yet another aspect of the present disclosure, various applications of a controlled release substance are provided. In one embodiment of a controlled release fabric care substance, the substance comprises at least one active fabric care substance in a host matrix comprising an organosilica gel. The fabric care substance may be at least one of a protein, enzyme, fragrance, anti-static agent, fabric softener, dye, or detergent, and may be in bead form, particle form or powder form. In another embodiment of controlled release personal care product, the product comprises at least one active personal care substance in a host matrix comprising an organosilica gel. The fabric care substance may be at least one of a protein, enzyme, fragrance, anti-static agent, fabric softener, dye, or detergent, and may be in bead form, particle form or powder form. The organosilica gels may be functionalized with amino groups.

Further areas of applicability will become apparent from the description provided herein. It should be understood that the description and specific examples are intended for purposes of illustration only and are not intended to limit the scope of the present disclosure.

### DETAILED DESCRIPTION

The following description is merely exemplary in nature and is not intended to limit the present disclosure, application, or uses.

According to various aspects of the present disclosure, there are provided various exemplary embodiments of a controlled release substance including an active agent disposed in a host matrix comprising an organosilica gel. In the various embodiments, the controlled release and delivery of active ingredients or agents is based on compositions of quick dissolving materials that can be used in applications where a fast, rapid and quick release of actives is important. The dissolution rate and time of these materials can be controlled and regulated by careful selection of the material compositions. Various controlled release materials were tested for release of actives in detergents. However, the controlled release compositions of the present application may be employed for multiple purposes, and used for a wide variety of controlled release applications.

The various embodiments of a controlled release substance are based on a design strategy that utilized organosilica gels whose properties can be tailored to elicit the desired controlled dissolution. The dissolution rate and application time of these materials can be efficiently controlled by the proper selection of the materials to yield dissolution times ranging from a few seconds to several minutes to several hours. Therefore, the controlled release substances utilizing organosilica gels can be used in a wide range of applications. The materials used in the various embodiments are also compatible with regular molecules as well as bio-molecules, and therefore can be used in generic release applications independent of the nature of the actives. The inventors believe the materials used in the various embodiments provide unprecedented flexibility in the design of controlled release substances, and constitutes a novel family of controlled release compositions and materials. The inventors are not aware of competing technologies that provide the range or flexibility in design to control the rate and time of dissolution that can be obtained with the organosilica sol-gels materials disclosed herein.

The various embodiments of controlled release compositions and materials are comprised of organosilica sol-gels functionalized with amino groups, which may be prepared by hydrolysis of aminosilane precursors. The aminosilane precursors may generally be prepared in solution. The various embodiments of materials and compositions comprising the organosilica sol-gel or aminosilane precursors are further aged, where a gelation process occurs by polymerization of the monomers to form particles, which grow in size and link to form chains followed by branched chains and finally formation of an extensive network. This extensive network may be physically seen as a gel. The gels are characterized by a loosely held network of particles with pore sizes on the order of 0.5 nm which is suitable for dissolution-controlled release. It is believed that hydrogen bonding holds the particles together. It is also believed that the formation of protonated amino groups due to individually electrostatic repulsions prevent formation of a network and facilitate dissolution when ghe gels are place in water. The following examples disclose the compositions and preparation of various embodiments of controlled release compositions of the present disclosure.

### EXAMPLE 1

One embodiment of a sol-gel (K1) was prepared with 0.5 mL of (3-aminopropyl) trimethoxysilane (2.86 mmol), 0.5 mL of [N-(2-aminoethyl)-3-aminopropyl]trimethoxysilane (2.23 mmol), and 0.4 mL (22.2 mmol) of H₂O.

### EXAMPLE 2

A second embodiment of a sol-gel (K2) was prepared with 1.0 mL of [3-(trimethoxysilyl) propyl]diethylenetriamine (3.67 mmol) and 0.4 mL of H₂O.

### EXAMPLE 3

A third embodiment of a sol-gel (K3) was prepared with 1.0 mL of (3-aminopropyl) trimethoxysilane (2.86 mmol), 0.5 mL of [N-(2-aminoethyl)-3-aminopropyl]trimethoxysilane (5.73 mmol), and 0.4 mL of H₂O.

### EXAMPLE 4

A fourth embodiment of a sol-gel (K4) was prepared with 0.5 mL of (3-aminopropyl) trimethoxysilane (2.86 mmol), 0.5 mL of [N,N-bis(2-hydroxyethyl)-3-aminopropyl]-trimethoxysilane (1.41 mmol), and 0.4 mL (22.2 6L mmol) of H₂O.

### EXAMPLE 5

A fifth embodiment of a sol-gel (K5) was prepared with 0.5 mL of [N-(hydroxyethyl)-N-methylamino-propyl]trimethoxysilane (1.58 mmol), (3-aminopropyl) trimethoxysilane (2.86 mmol), and 0.4 mL (22.2 mmol) of H₂O.

The second embodiment of sol-gel composition was initially optimized fro encapsulation and controlled release of Congo red dye molucles, due to the ease of monitoring the release of the colored dye molecule. The K2 sample took approximately 30 minutes to release the encapsulated dye when placed in 10 mL of water. The time take for dissolution of a constant measured among (0.1 g) of dye containing gels in 10 mL of water is shown in Table 1 of the paper “Controlled Dissolution of Organosilica Sol-Gels As A Means For Water-Regulated Release/Delivery Of Actives In Fabric Care Applications”, which is incorporated by reference herein in its entirety at the end of this disclosure.

Various embodiments of enzyme containing controlled release compositions and materials are also disclosed. The various embodiments of enzyme containing controlled release compositions and materials may be prepared using at least two methods. In the first method, the enzyme containing gels were prepared by the addition of formulated enzyme, immediately after addition of water to the siloxane precursor mixture. The assay of the enzyme released from these sol-gels were found to have significant loss of enzyme activity due to the heat released during the hydrolysis and condensation reactions of the siloxane precursors with water. This heating of the mixture is believed to be the cause of denaturation of the sol-gel. Mixing the enzyme and precursor along with water kept in an ice bath to form the gel samples were found to retain 100 percent of enzyme activity and prevent denaturation of enzymes.

In the second method, the silozane water mixture was allowed to “age” for 30-90 minutes prior to addition of the enzyme. On addition of water, trialkoxyaminosilanes readily undergo hydrolysis. Letting the sol-gel stand under ambient conditions also allows the sol to achieve equilibrium. When the pre-hydrolized sol of this method was used, a lesser degree of denaturation and better retention of native activity was achieved. Using pre-hydrolized sol prior to addition of the enzyme was also found to retain the enzyme activity upon encapsulation. The formulated enzyme-containing K1 and K2 gels seem to have comparatively less hydrogen-bonding interactions, and thus these gels were more easily dissolved than the pristine K1 and K2 gels themselves.

The results on release of dye and enzyme provided in the paper “Controlled Dissolution of Organosilica Sol-Gels As A Means For Water-Regulated Release/Delivery Of Actives In Fabric Care Applications” provide an indication of the utility of these materials in dissolution-controlled release. The quick dissolution times of 10 minutes found in sol-gels labeled K1, K3, K4 and K5 are ideal for fabric care applications where a rapid release during the washing process is necessary. The systems with only amino groups (K1, K2, K3) were found to have shorter gelation times as compared to hydroxyl groups (K4, K5). The system with the greatest number of amino groups in precursor (K2) realized the fastest gelation and slowest dissolution. On the other hand, systems containing precursors where both the hydrogen atoms on the terminal amino groups have been substituted with hydroxyethyl groups (K4) or with a hydroxyethyl and a methyl group (K5) realized the longest gelation time. Accordingly, reducing the number of amino groups or substitution of hydrogen on the amino group with a non-hydrogen-bonding methyl group lowers the dissolution times. Thus, the various embodiments of enzyme containing controlled release compositions and materials may be tailored to elicit controlled dissolution by proper selection of the materials. The above disclosed materials (and their equivalents) provide unprecedented flexibility in the design of controlled release substances, to control the rate and time of dissolution. The disclosed organosilica sol-gels are also biocompatible and nontoxic, which makes the material usable in routine consumer applications. One such example of a consumer application is a controlled release fabric care substance.

In yet another aspect of the present disclosure, various applications of a controlled release substance are provided. In one embodiment of a controlled release fabric care substance, the substance comprises at least one active fabric care substance in a host matrix comprising an organosilica gel. The organosilica gel may be functionalized with amino groups. The fabric care substance may be at least one of a protein, enzyme, fragrance, anti-static agent, fabric softener, dye, or detergent, and may be in bead form, particle form or powder form.

In another embodiment of controlled release personal care product, the product comprises at least one active personal care substance in a host matrix comprising an organosilica gel. The organosilica gel may be functionalized with amino groups. The fabric care substance may be at least one of a protein, enzyme, fragrance, anti-static agent, fabric softener, dye, or detergent, and may be in bead form, particle form or powder form.

It should be noted that the above described compositions and materials are exemplary in nature, and should not be construed to limit the scope of the invention. Additional design considerations, readily apparent to one of ordinary skill in the art, such as the substitution of hydroxyethyl groups or methyl groups may also improve the controlled dissolution rate of the materials. It should be apparent to those skilled in the art that various modifications such as the above may be made without departing from the spirit and scope of the invention. Accordingly, it is not intended that the invention be limited by the particular form illustrated and described above, but by the appended claims.

what is claimed is:

1. A controlled release fabric care substance comprising at least one active fabric care substance in a host matrix comprising an organosilica gel.

2. The controlled release fabric care substance according to claim 1, wherein the fabric care substance is at least one of a protein, enzyme, fragrance, anti-static agent, fabric softener, dye, or detergent.

3. The controlled release fabric care substance according to claim 2 wherein the substance is in bead form.

4. The controlled release fabric care substance according to claim 2 wherein the substance is in particle form.

5. The controlled release fabric care substance according to claim 2 wherein the substance is in powder form.

6. The controlled release fabric care substance according to claim 2 wherein the substance is in tablet form.

7. The controlled release fabric care substance according to claim 1 wherein the organosilica gel is functionalized with amino groups.

8. A controlled release personal care product comprising at least one active personal care substance in a host matrix comprising an organosilica gel.

9. The controlled release fabric care substance according to claim 8, wherein the personal care substance is at least one of a protein, enzyme, fragrance, emollient, moisturizer, tanning agent, uv protectant, or conditioner.

10. The controlled release fabric care substance according to claim 8 wherein the substance is in bead form.

11. The controlled release fabric care substance according to claim 8 wherein the substance is in particle form.

12. The controlled release fabric care substance according to claim 8 wherein the substance is in powder form.

13. The controlled release fabric care substance according to claim 8 wherein the substance is in tablet form.

14. A controlled release pharmaceutical preparation comprising at least one pharmacaolgically active substance in a host matrix comprising a biocompatible organosilica gel.

15. The controlled release fabric care substance according to claim 14 wherein the substance is in bead form.

16. The controlled release fabric care substance according to claim 14 wherein the substance is in particle form.

17. The controlled release fabric care substance according to claim 14 wherein the substance is in powder form.

18. The controlled release fabric care substance according to claim 14 wherein the substance is in tablet for
